(19) United States
(12) Patent Application Publication
(10) Pub. No.: US 2017/0151123 A1
(43) Pub. Date: Jun. 1, 2017

(54) REHABILITATION SYSTEM AND METHOD

(71) Applicant: KONINKLIJKE PHILIPS N.V., EINDHOVEN (NL)

(72) Inventors: Mirela Alina WEFFERS-ALBU, Boukoul (NL); Raymond VAN EE, Geldrop (NL)

(21) Appl. No.: 15/310,112

(22) PCT Filed: Jun. 3, 2015
(86) PCT No.: PCT/EP2015/001148

§ 371 Date: Nov. 10, 2016

(30) Foreign Application Priority Data
Jun. 3, 2014 (EP) 14170887.5

(51) Int. Cl. A61H 23/02; A61B 5/0488; A61B 5/00; A61F 7/00

(52) U.S. Cl. CPC A61H 23/02; A61F 7/00; A61B 5/0488; A61B 5/744; A61F 2007/0043

(57) ABSTRACT

The present invention relates to a rehabilitation system (10) for a patient (22) suffering from a damaged muscle and/or nerve, said system (10) comprising: a stimulation unit (12) for stimulating the damaged muscle and/or nerve; a display (16) for displaying a representation (30) of an affected body part of the patient (22) in which the damaged muscle and/or nerve is arranged; and a control unit (14) for controlling the display (16) to display, concurrently with a stimulation provided by the stimulation unit (12), a visual cue (28, 28′) depicted in the representation (30) of the affected body part, wherein said visual cue (28, 28′) visually indicates the stimulation of the damaged muscle and/or nerve.

### FIELD OF THE INVENTION

The present invention relates to a rehabilitation system for a patient suffering from a damaged muscle and/or nerve. The present invention particularly relates to a rehabilitation system using congruent multi-sensory stimulation. Further, the present invention relates to a corresponding method for rehabilitating a patient.

### BACKGROUND OF THE INVENTION

A general characteristic common to patients suffering from temporary paralysis due to trauma, hernia, longtime lying in bed, or just simply old age is the fact that although such patients may have a healthy neuro-activity necessary for limbic motion, their muscles and/or nerves are damaged.

Rehabilitation for damaged muscles and/or nerves is currently done mainly in rehabilitation centers where patients are under the care of physiatrists, rehab nurses, occupational therapists and physical therapists and undergo specific physiotherapy programs aiming at recovering the damaged element in the muscle and/or nerve. Physical therapists also use hot and/or cold packs as well as ultrasound systems which use high frequency waves to produce heat.

The costs associated with muscle nerve regeneration can be enormous. For instance, “according to statistics reported by the Foundation for Spinal Cord Injury Prevention and Cure, the lifetime medical costs for paraplegia are $1 million. The lifetime medical costs for quadriplegia range from $1.5 million to $3 million. The statistics do not include lost wages or other financial losses” (http://www.georgiaaccidentlaws.com/georgiaautoaccidentparalysislaws.html). Effective, but cost-saving rehabilitation systems for the home use are therefore desirable.

US 2011/0213266 A1 discloses a closed loop, neural activity triggered rehabilitation device and method for facilitating recovery of a patient from the effects of a sensory motor disability. The device includes a sensor system positionable adjacent the brain of the patient for detecting neural signals. A functional stimulation component is operatively connectable to at least one body part, such as a muscle or a nerve. The functional stimulation component stimulates the at least one body part in response to the neural signals detected. A sensory stimulation module is operatively connected to the patient to provide sensory feedback thereto.

While such closed loop systems using brain signals have shown advantageous recovery effects for the patients, the recovery of the patient still progresses very slowly, with many set-backs, and very small steps forward that are difficult, if not impossible, to perceive by the patient especially in the beginning. Not being able to perceive benefits of the treatment commonly leads to patient discouragement, lack of trust and adherence to the program or device, thereby impeding the rehabilitation process. Many patients expect a very fast recovery and if no such fast recovery may be perceived by the patient, they mistrust the functionality of the device. Known devices are often lacking to give the patients a feedback about the recovery process. User-adherence to such devices is therefore in most of the cases quite low.

Thus, there is still room for improvement.

US 2006/0195042 A1 discloses a system and method for a biological interface system that processes multicellular signals of a patient and controls one or more devices. The system includes a sensor that detects the multicellular signals and a processing unit for producing the control signal based on the multicellular signals. The system further includes an automated configuration routine that is used to set or modify the value of one or more system configuration parameters.

### SUMMARY OF THE INVENTION

It is an object of the present invention to provide a rehabilitation system for a patient suffering from damaged muscles and/or nerves which substantially alleviates or overcomes the above mentioned problems. In particular, it is an object of the present invention to provide a rehabilitation system which is applicable for home use and improves the user-adherence compared to known devices of this type. It is furthermore an object to provide a corresponding rehabilitation method.

According to a first aspect of the present invention, a rehabilitation system for a patient suffering from a damaged muscle and/or nerve is presented, wherein the system comprises:

a stimulation unit for stimulating the damaged muscle and/or nerve;

a display for displaying a representation of an affected body part of the patient in which the damaged muscle and/or nerve is arranged; and

a control unit for controlling the display to display, concurrently and congruently with a stimulation provided by the stimulation unit, a visual cue depicted in the representation of the affected body part, wherein said visual cue visually indicates the stimulation of the damaged muscle and/or nerve and is provided simultaneously and adapted to the stimulation of the damaged muscle and/or nerve.

According to a second aspect of the present invention, a method for rehabilitating a patient suffering from a damaged muscle and/or nerve is provided, wherein the method comprises the steps of:

stimulating the damaged muscle and/or nerve of the patient;

displaying a representation of an affected body part of the patient in which the damaged muscle and/or nerve is arranged; and

displaying a visual cue depicted in the representation of the affected body part concurrently and congruently to stimulating it, said visual cue indicating the stimulation of the damaged muscle and/or nerve and being provided simultaneously and adapted to the stimulation of the damaged muscle and/or nerve.

Preferred embodiments of the invention are defined in the dependent claims. It shall be understood that the claimed method has similar and/or identical preferred embodiments as the claimed system and as defined in the dependent claims.

In contrast to known devices of this kind, the presented rehabilitation system does not only stimulate the damaged muscle and/or nerve, but additionally visualizes the stimulation provided by the stimulation unit on a display. Even if the patient does not recognize the stimulation of his/her muscles and/or nerves, he/she gets a visual feedback via the display that the stimulation is ongoing as well as a feedback to which part of his/her body the stimulation is provided. The patient may also get at least a visual feedback about the progression of the recovery of the treated muscles and/or nerves. Thus, the patient so to say receives a multi-sensorial stimulation, a tactile stimulation provided by the stimulation unit and a visual stimulation provided concurrently and congruently via the display.

The control unit thereto controls the display to display a representation of the affected body part of the patient. This representation may not only include the affected body part, but also the whole body of the patient. As it will be elucidated further below, the control unit may control the display to display an avatar mirroring the patient. In addition to this representation, the control unit controls the display to display visual cues directly within the graphical representation of the affected body part. These visual cues provide a visual feedback to the patient and show him/her the ongoing stimulation of the damaged muscle and/or nerve in a graphical way.

The terms “concurrently” and/or “congruently” shall mean that the visual stimulation provided by the visual cues on the display and the tactile stimulation provided by the stimulation unit are provided simultaneously and are adapted to one another. The formulation that the “visual cue [is] depicted in the representation of the affected body part” shall mean that the visual cue is depicted at or on the representation of the affected body part. The visual cue is thus not provided separately to the representation of the affected body part, but together with it in one and the same graphical representation on the display.

Such a multi-sensorial stimulation of the patient provides several advantages: The brain is continuously looking for associations and tries to find correlations between brain activity and muscular activity. A well-known statement is “firing together is wiring together”. This means that if the brain detects that a stimulation from the environment evokes neural activity in two groups of neurons, then the two groups of neurons will be connected. If the stimulation provided to the muscles and/or nerves by the stimulation unit are concurrently graphically shown (e.g. in a schematic manner), such an additional visualization may strengthen the correlations within the brain, thereby generating increased and ongoing stimulation of the nerves, and may thus significantly help to improve the rehabilitation success. By showing these visual cues within the representation of the affected body part, the visual indication becomes much more realistic for the patient, since the patient may think that the display in fact shows his/her body.

A further significant advantage of the multi-sensorial approach followed in the present invention is that the patient may at least visually perceive the ongoing stimulation even if he/she does not tangibly perceive the stimulation on his/her body. This might especially be helpful in early stages of the rehabilitation therapy. Patients may in this way perceive progress of the therapy even before their impaired limbs including the damaged muscles and/or nerves are able to actually move. Hence, patients may stay motivated to adhere to the therapy program, such that the chance of rehabilitation success is enhanced.

According to an embodiment, the stimulation unit comprises one or more actuators for generating a tactile stimulation of the damaged muscle and/or nerve. These tactile actuators are preferably configured to provide a time-variable tactile stimulus. The one or more tactile actuators may, for example, comprise one or more piezoelectric elements.

According to a preferred embodiment, the stimulation unit comprises a body attachment component for attaching the stimulation unit to the affected body part. The stimulation unit may thus be realized as a portable, wearable component. This results in a non-invasive device that disturbs the patient as few as possible.

The body attachment component preferably comprises one of a belt, strap, band, gear or harness that is configured to be worn on a limb of the patient. For example, the body attachment component may be realized as a stretchable strap that may be worn on a leg or an arm of the patient.

According to a further embodiment, the one or more actuators of the stimulation unit are arranged at or embedded into the body attachment component. In such a case, the wiring of the device is reduced as much as possible. The patient might not even recognize the actuators in this case, since they might not be visible form the exterior. This increases the comfort for the patient.

Each of the one or more actuators of the stimulation unit may comprise at least one vibration element for stimulating the damaged muscle and/or nerve by means of vibration. Alternatively or additionally, each actuator may comprise at least one thermo element for stimulating the damaged muscle and/or nerve by means of heat and/or cold. A concurrent stimulation by means of vibration and heat or cold often leads to the best rehabilitation results.

According to a further embodiment, the rehabilitation system may comprise a sound generation unit for generating a sound, wherein the control unit is configured to control the sound generation unit to generate a sound, concurrently with the stimulation provided by the stimulation unit, wherein said sound indicates the stimulation of the damaged muscle and/or nerve in audible form.

In this case, the stimulation is provided synchronously and congruently in three different ways: in tactile form by means of the stimulation unit, in visual form by means of the display, and in audible form by means of the sound generation unit. The stimulation unit may thus render a tactile stimulus (vibration and/or heat or cold), wherein said tactile stimulus is augmented by visual cues rendered on the display indicating the vibration and/or heat or cold, as well as corresponding vibration sounds generated by the sound generation unit. Such a stimulation using three different sensorial stimuli (tactile, visual and audial) may maximize the rehabilitation effect as well as it may enhance the user-adherence to the system. In case the patient does not feel the tactile stimulus especially at the beginning of the rehabilitation process, he/she at least perceives the visual and audible stimulus, such that the correlations between brain activity and muscular activity may be further improved.

According to a further embodiment, the control unit is configured to control the display to adapt the visual cue based on an intensity and/or type of stimulation provided by the stimulation unit.

Depending on the type of tactile stimulus that is provided by the stimulation unit, the visual cue that is visualized on the display may change. For example, if stronger vibrations are provided by the stimulation unit, the visual cue may be larger or have another appearance as if the patient is stimulated with weaker vibrations. If the stimulation unit renders a time-variable vibration stimulus, this may lead to another graphical representation of the visual cue than if the stimulation unit renders a time-constant vibration stimulus. The visual cues may also be provided in different colors depending on the type of stimulation provided by the stimulation unit. In case the stimulation unit renders warm temperature stimuli, the tactile stimulation may be augmented by red visual cues depicted in the corresponding representation of the affected body part, indicating a higher temperature. If, on the other hand, the stimulation unit renders cold temperature stimuli, the tactile stimulation may be augmented by blue visual cues depicted in the display on the representation of the affected body part, indicating lower temperature. Thus, various degrees of vibrations and/or temperatures may be depicted in the display by various types and/or colors of the visual cues.

In a similar way, the control unit is preferably configured to control the sound generation unit to adapt the sound based on an intensity and/or type of stimulation provided by the stimulation unit. The timing of the generated sounds is thus adapted to the timing of the tactile stimuli. In case the tactile stimuli change, the sounds may change as well. For example, if the tactile stimuli get stronger, the sounds may get louder.

In a further embodiment, the control unit is configured to control the display to display an avatar, wherein the avatar includes the representation of the affected body part. Such an avatar appears to be more realistic for the patient than just simply showing a picture of the affected body part on the display.

In a refinement, the rehabilitation system may further comprise an input interface for receiving anatomical information of the patient, wherein the control unit is configured to control the display to adapt the avatar based on the anatomical information of the patient.

In this case, the visualized avatar so to say mirrors the “real” patient's appearance in a realistic way. The avatar may, for example, be visualized in real-size on the display. The above-mentioned input interface allows to input a picture of the patient or his/her face as well as to input the height, weight and/or other anatomical characteristics of the patient, such that the control unit may generate an avatar that resembles the appearance of the patient best.

According to a further embodiment, the rehabilitation system may further comprise a sensory unit with at least one electromyography (EMG) sensor for sensing a response of the damaged muscle and/or nerve to the stimulation provided by the stimulation unit.

This EMG sensor may be arranged at or embedded into the body attachment component into which the at least one vibration element and/or the at least one thermo element is embedded as well. The provision of such an EMG sensor enables to measure the response of the muscle and/or nerve to the provided stimuli. The control unit may in this case be configured to control the display to display an indication of the response of the damaged muscle and/or nerve. In other words, it is thereby possible to give the patient a feedback on the display regarding the trend of the muscle and/or nerve recovery. For example, statistics over such trend's may be displayed in addition to the representation of the affected body part. This may further increase the user-adherence to the system, as the patient may directly observe the benefits of the therapy.

### BRIEF DESCRIPTION OF THE DRAWINGS

These and other aspects of the invention will be apparent from and elucidated with reference to the embodiments described hereinafter. In the following drawings

FIG. 1A shows a schematic view of a first embodiment of a rehabilitation system according to the present invention;

FIG. 1B shows a schematic view of a stimulation unit of the rehabilitation system according to the first embodiment;

FIG. 2A shows a schematic view of a second embodiment of the rehabilitation system according to the present invention;

FIG. 2B shows a schematic view of the stimulation unit of the rehabilitation system according to the second embodiment; and

FIG. 3 shows a schematic diagram illustrating a method for rehabilitating a patient according to the present invention.

### DETAILED DESCRIPTION OF THE INVENTION

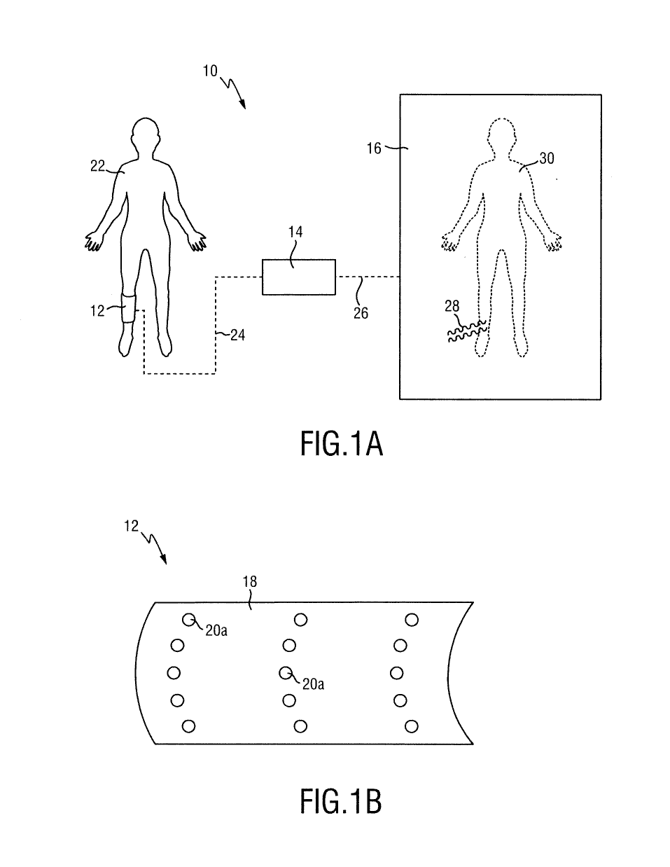

FIG. 1A shows a first embodiment of a rehabilitation system according to the present invention. The rehabilitation system is therein denoted in its entirety with reference numeral 10.

The rehabilitation system 10 comprises a stimulation unit 12, a control unit 14 and a display 16. An enlarged schematic view of an exemplary stimulation unit 12 is shown in FIG. 1B.

The stimulation unit 12 may according to the first embodiment comprise a body attachment component and a plurality of actuators 20a that are configured to generate a tactile stimulus for stimulating a damaged muscle and/or nerve of a patient 22. These actuators 20a may be arranged at the body attachment component 18 or may even be embedded into the body attachment component 18. The actuators 20a itself may be realized as vibration elements. The actuators 20a may thereto comprise one or more piezoelectric elements. However, it shall be noted that vibratory actuators other than piezoelectric elements may be used without leaving the scope of the present invention.

The body attachment component 18 is realized as a wearable component that may be worn on one of the limbs of the patient 22 i.e. on an arm or a leg of the patient 22. According to a preferred embodiment, said body attachment component 18 comprises one of a belt, strap, band, gear or harness that is made of an elastic material, such that the body attachment component 18 fits to an arm as well as to a leg of the patient 22, wherein the patient does not have to manually adapt its size.

The control unit 14 may be realized as a processor, microprocessor or computer having software stored thereon for controlling the display 16 according to the principle of the present invention, as this will be elucidated in detail further below.

The display 16 may comprise a screen, such as a monitor with a cathode ray tube, a LCD monitor, a plasma monitor or any other type of monitor. Alternatively, the display 16 may comprise a projector or beamer for projecting images on to a wall or screen.

The control unit 14 is connected to the stimulation unit 12 as well as to the display 16. The connection 24 between the stimulation unit 12 and the control unit 14 as well as the connection 26 between the control unit 14 and the display 16 may either be hard-wired or wireless (e.g. using a Bluetooth®, an infrared, a WLAN or near field communication technique). Even though the control unit 14 is in FIG. 1A shown as a separate unit that is arranged remotely from the stimulation unit 12 and the display 16, parts of the control unit 14 or the whole control unit 14 may be integrated into or arranged at the stimulation unit 12 or the display 16.

The presented system 10 addresses patients 22 with a generally healthy neuro-activity necessary for limbic motion, but damaged muscles and/or muscles nerves. The system 10 should help such patients 22 to revitalize the muscle tonus and/or muscle nerves necessary for limbic motion.

A central feature of the system 10 according to the present invention is a congruent multi-sensory stimulation of the patient 22 that shall improve the rehabilitation process of the patient's muscles and/or nerves. In contrast to known systems of this type, the presented system 10 does not only stimulate the affected body part of the patient 22 by means of tactile stimuli, but concurrently provides a visual stimulation to the patient 22. The control unit 14 thereto controls the stimulation unit 12 and the display 16 in a congruent manner. The control unit 14 in other words controls the display 16 to display a visual cue 28 concurrently and congruently with a stimulation provided by the stimulation unit 12. This visual cue 28 is displayed together with a graphical representation 30 of the patient 22. The visual cue 28 visually indicates the stimulation of the damaged muscle and/or nerve to the patient 22.

In a preferred embodiment, the display 16 is configured to display an avatar that mirrors the anatomical appearance of the patient 22. However, in some cases it might also be sufficient to display only a graphical representation of the affected body part of the patient 22 on the display 16.

The idea of additionally indicating the stimulation in visual form on the display 16 is to stimulate the different senses of the patient 22 concurrently. Such a multi-sensorial stimulation is especially advantageous at the beginning of the recovery process when the patient 22 might not perceive the tactile stimuli provided by the stimulation unit 12. In this case, the patient 22 at least receives a visual indication on the display 16 that gives him/her a feedback regarding the provided stimulation. This may increase the user-adherence and motivation of the patient 22 as well as it may enhance the recovery process. If the patient 22 perceives the tactile stimulation provided by the stimulation unit 12 concurrently with the visual stimulation provided by the visual cues 28 displayed on the display 16, the brain of the patient 22 may recognize a correlation that may enhance the recovery of the affected muscle and/or nerve.

The control unit 14 is preferably configured to adapt the appearance (e.g. the shape and/or size) of the visual cue 28 depending on the type and/or intensity of the tactile stimuli provided by the actuators 20a. The visual cue 28 may include one or more graphical symbols or pictograms which change dynamically depending on the stimuli provided by the actuators 20a. The visual cues may also include video cues, such as smaller video sequences. These video cues may, for example, be shown with the same fade-in frequency as the frequency of the tactile stimuli provided by the actuators 20a. If the stimuli provided by the actuators 20a get stronger, the visual cues 28 may get larger.

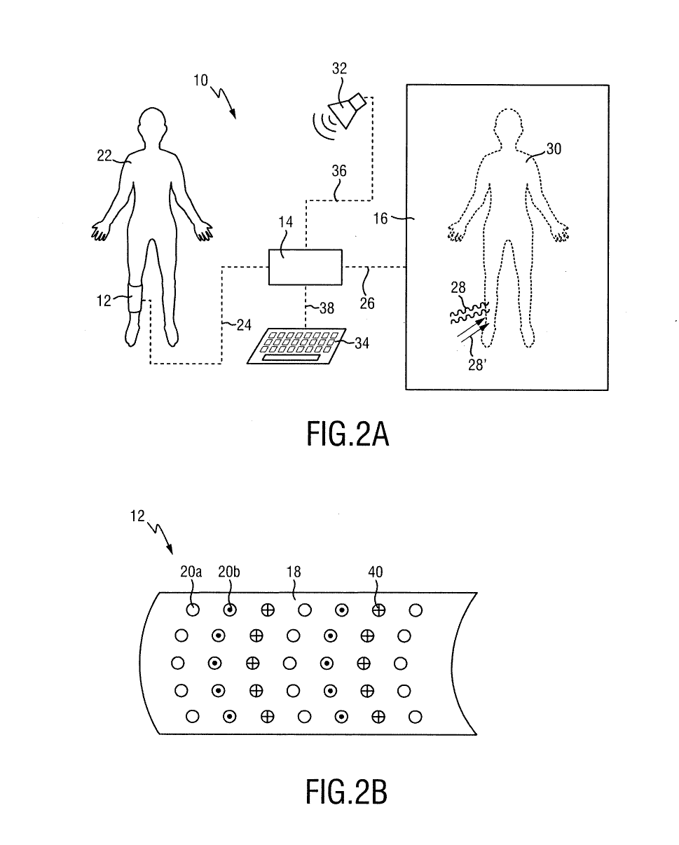

FIGS. 2A and 2B show a second embodiment of the system 10 according to the present invention. Same or similar components are therein denoted by the same reference numerals as before. These already presented components shall not be explained again.

Additionally to the components of the system 10 according to the first embodiment, the system 10 according to the second embodiment further comprises a sound generation unit 32 and an input interface 34. Both the sound generation unit 32 and the input interface 34 are connected to the control unit 14. The connection 36 between the sound generation unit 32 and the control unit 14 as well as the connection 38 between the input interface 34 and the control unit 14 may again be realized either as a hard-wired connection or as a wireless connection.

The sound generation unit 32 may, for example, comprise a loudspeaker or any other type of device for generating sounds. The input interface 34 may comprise a keyboard, mouse, an USB-interface or any other type of data interface that allows a user to input or transfer data to the control unit 14. The input interface 34 may also be used to adapt the settings of the control unit 14.

The addition of the sound generation unit 32 enables to produce sounds congruently with the tactile stimuli provided by the stimulation unit 12 and the visual cues 28 displayed on the display 16. The sensorial stimulation is thus expanded to one additional stimulus (an audible stimulus). The control unit 14 is thereto configured to control the sound generation unit 32 to produce sounds that are adapted to the stimuli provided by the stimulation unit 12. The produced sounds are preferably adapted based on an intensity and/or type of stimulation provided by the stimulation unit 12.

As shown in FIG. 2B, the stimulation unit 12 may furthermore comprise one or more thermo elements 20b for stimulating the damaged muscle and/or nerve by means of heat and/or cold. The control unit 14 may be configured to control the display 16 to display additional visual cues 28′ within the representation 30 of the patient 22 in order to visually indicate the stimuli provided by the thermo elements 20b. These visual cues 28′ are also preferably adapted to the intensity and/or type of the stimuli provided by the thermo elements 20b. In case the control unit controls the stimulation unit 12/the thermo elements 20b to render warm temperature stimuli, this stimulation may be augmented by glowing red visual cues 28′ depicted in the visual representation 30 of the patient 22 within the region of the affected body part. In case the control unit 14 controls the stimulation unit 12/the thermo elements 20b to render cold temperature stimuli, this stimulation may be augmented by blue visual cues 28′ depicted in the representation 30 of the patient 22 within the region of the affected body part. Various degrees of temperature may be depicted by various intensity degrees of red and/or blue.

The stimulation unit 12 may furthermore comprise one or more EMG sensors 40 for sensing a response of the stimulated muscle and/or nerve to the stimulation provided by the stimulation unit 12. Sensing the response of the stimulated muscle and/or nerve by means of EMG sensors 40 allows displaying an indication of said muscle and/or nerve response on the display 16. This visualization may again be controlled by the control unit 14. The patient 12 may, for example, be shown a graphical symbol or graphical signal evaluation indicating the sensorial response of the damaged muscle and/or nerve to the stimulation via the display 16. The display 16 may also graphically show information regarding the tendency and/or prospects of the muscle and/or nerve recovery.

In order to provide an even more realistic feeling for the patient 22, the graphical representation 30 shown on the display 16 may include an avatar, wherein the avatar mirrors the patient 22 in a graphical form as realistic as possible. The input interface 34 may, for example, be used to input a picture of the patient 22, a height of the patient 22, a weight of the patient 22 and/or an indication of the built of the patient 22 (lean/medium/heavy), such that the avatar shown on the display 16 resembles the patient 22 as close as possible. In other words, the control unit 14 may be configured to control the display 16 to adapt the graphical representation 30 based on the anatomical information of the patient 22 that are received via the input interface 34.

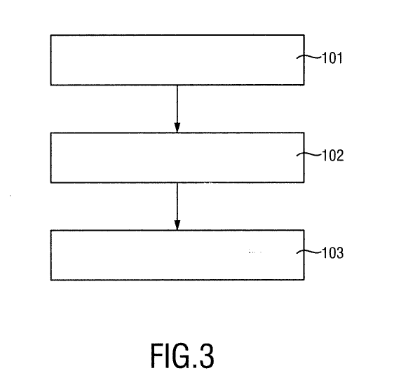

FIG. 3 summarizes the method according to the present invention in a schematic manner. In a first step 101, the damaged muscles and/or nerves of the patient 22 are stimulated by means of a tactile stimulation. In a second step, a representation of the affected body part of the patient 22 in which the damaged muscle and/or nerve is arranged is displayed on a display 16. In a third step 103, a visual cue 28 is displayed on the representation of the affected body part concurrently to the provided tactile stimulation. Said visual cue 28, 28′ visually indicates the stimulation of the damaged muscles and/or nerves and therefore provide a visual stimulation that is provided to the patient in a congruent manner to the tactile stimulation. As explained before, this multi-sensorial stimulation may be furthermore enhanced by congruently generating sounds that are adapted to the tactile stimuli.

It shall be noted that FIG. 3 shows the presented method for simplicity reasons in a sequential form. However, steps 101-103 are in practice carried out concurrently.

Lastly, it shall be also noted that the components of the second embodiment of the system 10, which were not elucidated with reference to the first embodiment shown in FIG. 1, do not necessarily have to be provided all together. For example, the sound generation unit 32 and the input interface 34 are independent parts. The system 10 may therefore also comprise only one of these components. The stimulation unit 12 may similarly comprise only one of the vibratory actuators 20a and the thermo elements 20b.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. A rehabilitation system for a patient suffering from damaged muscles and/or nerves, said system comprising:
a stimulation unit for stimulating a damaged muscle and/or nerve;
a display for displaying a representation of an affected body part of the patient in which the damaged muscle and/or nerve is arranged; and
a control unit for controlling the display to display, concurrently and congruently with a stimulation provided by the stimulation unit, a visual cue depicted in the representation of the affected body part, wherein said visual cue visually indicates the stimulation of the damaged muscle and/or nerve that is provided simultaneously to the stimulation and dynamically adapted based on an intensity and/or type of the stimulation of the damaged muscle and/or nerve.

2. The rehabilitation system according to claim 1, wherein the stimulation unit comprises an actuator for generating a tactile stimulation of the damaged muscle and/or nerve.

3. The rehabilitation system according to claim 1, wherein the stimulation unit comprises a body attachment component for attaching the stimulation unit to the affected body part.

4. The rehabilitation system according to claim 3, wherein the body attachment component comprises one of a belt, strap, band, gear or harness that is configured to be worn on a limb of the patient.

5. The rehabilitation system according to claim 3, wherein the actuator is arranged at or embedded into the body attachment component.

6. The rehabilitation system according to claim 2, wherein the actuator comprises at least one vibration element for stimulating the damaged muscle and/or nerve by means of vibration.

7. The rehabilitation system according to claim 2, wherein the actuator comprises at least one thermo element for stimulating the damaged muscle and/or nerve by means of heat and/or cold.

8. The rehabilitation system according to claim 1, further comprising a sound generation unit for generating a sound, wherein the control unit is configured to control the sound generation unit to generate a sound, concurrently with the stimulation provided by the stimulation unit, wherein said sound indicates the stimulation of the damaged muscle and/or nerve in audible form.

9. (canceled)

10. The rehabilitation system according to claim 8, wherein the control unit is configured to control the sound generation unit to adapt the sound based on an intensity and/or type of stimulation provided by the stimulation unit.

11. The rehabilitation system according to claim 1, wherein the control unit is configured to control the display to display an avatar, wherein the avatar includes the representation of the affected body part.

12. The rehabilitation system according to claim 11, further comprising an input interface for receiving anatomical information of the patient, wherein the control unit is configured to control the display to adapt the avatar based on the anatomical information of the patient.

13. The rehabilitation system according to claim 1, further comprising a sensor unit with at least one electromyography (EMG) sensor for sensing a response of the damaged muscle and/or nerve to the stimulation provided by the stimulation unit.

14. The rehabilitation system according to claim 1, wherein the control unit is configured to control the display to display an indication of the response of the damaged muscle and/or nerve.

15. Method for rehabilitating a patient suffering from damaged muscles and/or nerves, said method comprising the steps of:
stimulating a damaged muscle and/or nerve of the patient;
displaying a representation of an affected body part of the patient in which the damaged muscle and/or nerve is arranged; and
displaying a visual cue depicted in the representation of the affected body part concurrently and congruently to stimulating it, said visual cue indicating the stimulation of the damaged muscle and/or nerve that is provided simultaneously to the stimulation and dynamically adapted based on an intensity and/or type of the stimulation of the damaged muscle and/or nerve.
